(19) United States
(12) Patent Application Publication
(10) Pub. No.: US 2018/0028062 A1
(43) Pub. Date: Feb. 1, 2018

(54) ENDOSCOPE PROCESSOR

(71) Applicant: OLYMPUS CORPORATION, Tokyo (JP)

(72) Inventors: Soichiro KOSHIKA, Tokyo (JP); Kazuma KANEKO, Tokyo (JP)

(73) Assignee: OLYMPUS CORPORATION, Tokyo (JP)

(21) Appl. No.: 15/655,157

(22) Filed: Jul. 20, 2017

(30) Foreign Application Priority Data
Jul. 26, 2016 (JP) 2016-146254

(51) Int. Cl. A61B 5/00; A61B 1/00; A61B 1/07

(52) U.S. Cl. CPC A61B 5/0084; A61B 1/07; A61B 1/00172; A61B 1/0638

(57) ABSTRACT

An endoscope processor is an endoscope processor used in combination with a scanning type endoscope capable of scanning an object by displacing an irradiation position of illumination light to be radiated to the object, and includes: an image generation portion configured to respectively generate a plurality of color images according to return light of the illumination light radiated to the object; a correction processing portion configured to perform processing of acquiring a correction magnification for correcting a scanning width or a view angle of the scanning type endoscope in accordance with a reference value; and an image correction portion configured to perform magnification chromatic aberration correction processing for correcting a magnification chromatic aberration among the plurality of color images uniformly scaled according to the correction magnification.

### CROSS REFERENCE TO RELATED APPLICATION

This application claims the benefit of Japanese Application No. 2016-146254 filed in Japan on Jul. 26, 2016, the contents of which are incorporated herein by this reference.

### BACKGROUND OF THE INVENTION

### 1. Field of the Invention

The present invention relates to an endoscope processor, and in particular relates to an endoscope processor used in combination with a scanning type endoscope configured to optically scan an object.

### 2. Description of the Related Art

In an endoscope in a medical field, in order to reduce burdens on a subject, various technologies for narrowing a diameter of an insertion portion to be inserted into a body cavity of the subject have been proposed. Then, as one example of such technologies, a scanning type endoscope not including a solid-state image pickup device at a part corresponding to the insertion portion described above is known.

More specifically, a system including the scanning type endoscope is configured, for example, to transmit illumination light emitted from a light source by an optical fiber for illumination, two-dimensionally scan an object in a predetermined scanning route by driving an actuator for swinging a distal end portion of the optical fiber for illumination, receive return light from the object by an optical fiber for light reception, and generate an image of the object based on the return light received by the optical fiber for light reception. Then, for example, Japanese Patent No. 5490331 discloses an endoscope system similar to such a configuration.

More specifically, Japanese Patent No. 5490331 discloses an endoscope system including a scanning type endoscope, configured to use optical characteristic information of a predetermined objective optical system provided in the scanning type endoscope, and correct a magnification chromatic aberration generated due to the predetermined objective optical system.

### SUMMARY OF THE INVENTION

An endoscope processor of one aspect of the present invention is an endoscope processor used in combination with a scanning type endoscope capable of scanning an object by displacing an irradiation position of illumination light to be radiated to the object, and includes: an image generation portion configured to respectively generate a plurality of color images according to return light of the illumination light radiated to the object; a correction processing portion configured to perform processing of acquiring a correction magnification for correcting a scanning width or a view angle of the scanning type endoscope in accordance with a reference value; and an image correction portion configured to perform magnification chromatic aberration correction processing for correcting a magnification chromatic aberration among the plurality of color images uniformly scaled according to the correction magnification.

### BRIEF DESCRIPTION OF THE DRAWINGS

FIG. 1 is a diagram illustrating a configuration of a main portion of an endoscope system including an endoscope processor relating to an embodiment;

FIG. 2 is a sectional view for describing a configuration of an actuator portion;

FIG. 3 is a diagram illustrating one example of a signal waveform of a drive signal supplied to the actuator portion;

FIG. 4 is a diagram illustrating one example of a spiral scanning route from a center point A to an outermost point B;

FIG. 5 is a diagram illustrating one example of a spiral scanning route from the outermost point B to the center point A;

FIG. 6 is a diagram for describing an outline of table data used in processing of the endoscope processor relating to the embodiment;

FIG. 7 is a diagram for describing a specific example of a calculation method of a scanning width of an endoscope; and

FIG. 8 is a diagram illustrating one example of a test chart available when acquiring the scanning width of the endoscope.

### DETAILED DESCRIPTION OF THE PREFERRED EMBODIMENT

Hereinafter, the embodiment of the present invention will be described with reference to the drawings.

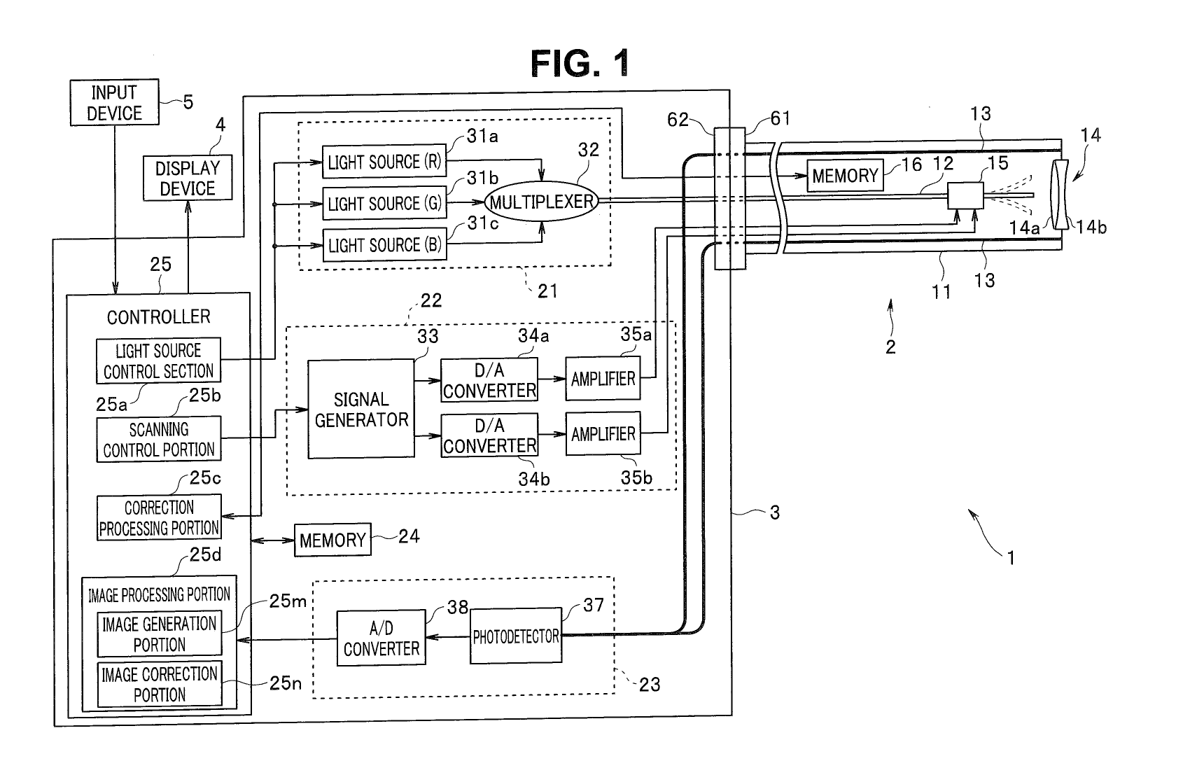

FIG. 1 to FIG. 8 relate to the embodiment of the present invention.

An endoscope system 1 is configured, for example, as illustrated in FIG. 1, including a scanning type endoscope (abbreviated simply as an endoscope, hereinafter) 2 to be inserted into a body cavity of a subject, a main body device 3 to which the endoscope 2 can be attachably and detachably connected, a display device 4 connected to the main body device 3, and an input device 5 capable of inputting information and giving an instruction to the main body device 3. FIG. 1 is a diagram illustrating a configuration of a main portion of the endoscope system including the endoscope processor relating to the embodiment.

The endoscope 2 is configured including an insertion portion 11 formed having an elongated shape insertable into a body cavity of a subject.

On a proximal end portion of the insertion portion 11, a connector portion 61 for attachably and detachably connecting the endoscope 2 to a connector receiving portion 62 of the main body device 3 is provided.

Inside the connector portion 61 and the connector receiving portion 62, though not shown in the figure, an electric connector device for electrically connecting the endoscope 2 and the main body device 3 is provided. In addition, inside the connector portion 61 and the connector receiving portion 62, though not shown in the figure, an optical connector device for optically connecting the endoscope 2 and the main body device 3 is provided.

To a part from the proximal end portion to a distal end portion inside the insertion portion 11, a fiber 12 for illumination which is an optical fiber configured to guide illumination light supplied from a light source unit 21 of the main body device 3 and emit the illumination light from an emission end portion, and a fiber 13 for light reception including one or more optical fibers for receiving return light from an object and guiding the return light to a detection unit 23 of the main body device 3 are inserted respectively. That is, the fiber 12 for illumination is configured having a function as a light guide portion.

An incident end portion including a light incident surface of the fiber 12 for illumination is arranged at a multiplexer 32 provided inside the main body device 3. In addition, the emission end portion including a light emission surface of the fiber 12 for illumination is arranged near a light incident surface of a lens 14a provided on the distal end portion of the insertion portion 11.

An incident end portion including a light incident surface of the fiber 13 for light reception is fixed and arranged around a light emission surface of a lens 14b on a distal end face of the distal end portion of the insertion portion 11. In addition, an emission end portion including a light emission surface of the fiber 13 for light reception is arranged at a photodetector 37 provided inside the main body device 3.

An illumination optical system 14 is configured including the lens 14a on which the illumination light through the light emission surface of the fiber 12 for illumination is made incident, and the lens 14b that emits the illumination light through the lens 14a to the object.

In a middle portion of the fiber 12 for illumination on a distal end portion side of the insertion portion 11, an actuator portion 15 driven based on a drive signal supplied from a driver unit 22 of the main body device 3 is provided.

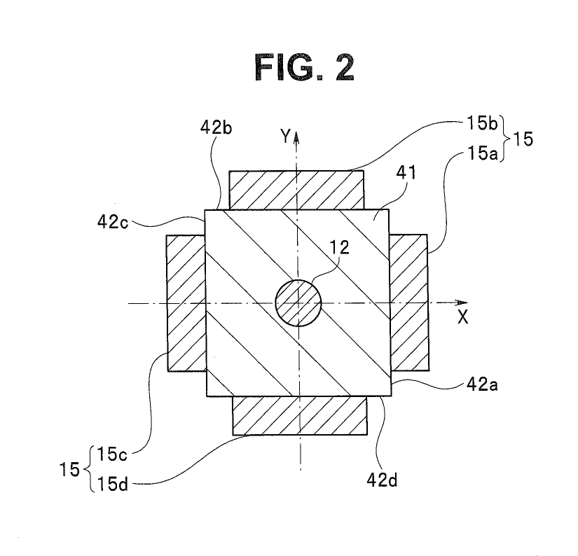

The fiber 12 for illumination and the actuator portion 15 are arranged respectively so as to have a position relation illustrated in FIG. 2, for example on a cross section vertical to a longitudinal axial direction of the insertion portion 11. FIG. 2 is a sectional view for describing a configuration of the actuator portion.

Between the fiber 12 for illumination and the actuator portion 15, as illustrated in FIG. 2, a ferrule 41 as a bonding member is arranged. More specifically, the ferrule 41 is formed by zirconia (ceramic) or nickel, for example.

The ferrule 41 is, as illustrated in FIG. 2, formed as a square pole, and includes side faces 42a and 42c vertical to an X axis direction which is a first axial direction orthogonal to the longitudinal axial direction of the insertion portion 11, and side faces 42b and 42d vertical to a Y axis direction which is a second axial direction orthogonal to the longitudinal axial direction of the insertion portion 11. In addition, at a center of the ferrule 41, the fiber 12 for illumination is fixed and arranged.

The actuator portion 15 includes, for example, as illustrated in FIG. 2, a piezoelectric element 15a arranged along the side face 42a, a piezoelectric element 15b arranged along the side face 42b, a piezoelectric element 15c arranged along the side face 42c, and a piezoelectric element 15d arranged along the side face 42d.

The piezoelectric elements 15a to 15d have polarization directions individually set beforehand, and are configured to expand and contract respectively according to a drive voltage applied by the drive signal supplied from the main body device 3.

That is, the piezoelectric elements 15a and 15c of the actuator portion 15 are configured as an actuator for an X axis capable of swinging the fiber 12 for illumination in the X axis direction by vibrating according to the drive signal supplied from the main body device 3. Furthermore, the piezoelectric elements 15b and 15d of the actuator portion 15 are configured as an actuator for a Y axis capable of swinging the fiber 12 for illumination in the Y axis direction by vibrating according to the drive signal supplied from the main body device 3.

Inside the insertion portion 11, a nonvolatile memory 16 that stores information including a scanning width Wa used in processing to be described later, for example, as intrinsic endoscope information for each endoscope 2 is provided. Then, the endoscope information stored in the memory 16 is read by a controller 25 of the main body device 3 when the connector portion 61 of the endoscope 2 and the connector receiving portion 62 of the main body device 3 are connected and a power source of the main body device 3 is turned on.

The main body device 3 is configured having a function as the endoscope processor. More specifically, the main body device 3 is configured including the light source unit 21, the driver unit 22, the detection unit 23, a memory 24, and the controller 25.

The light source unit 21 is configured including a light source 31a, a light source 31b, a light source 31c, and the multiplexer 32.

The light source 31a includes a laser light source for example, and is configured to emit light of a red wavelength band (also called R light, hereinafter) to the multiplexer 32 when the light is emitted by control of the controller 25.

The light source 31b includes a laser light source for example, and is configured to emit light of a green wavelength band (also called G light, hereinafter) to the multiplexer 32 when the light is emitted by the control of the controller 25.

The light source 31c includes a laser light source for example, and is configured to emit light of a blue wavelength band (also called B light, hereinafter) to the multiplexer 32 when the light is emitted by the control of the controller 25.

The multiplexer 32 is configured to multiplex the R light emitted from the light source 31a, the G light emitted from the light source 31b, and the B light emitted from the light source 31c, and supply the light to the light incident surface of the fiber 12 for illumination.

The driver unit 22 is configured to generate and supply a drive signal DA for driving the actuator for the X axis of the actuator portion 15 based on the control of the controller 25. In addition, the driver unit 22 is configured to generate and supply a drive signal DB for driving the actuator for the Y axis of the actuator portion 15 based on the control of the controller 25. Furthermore, the driver unit 22 is configured including a signal generator 33, D/A converters 34a and 34b, and amplifiers 35a and 35b.

The signal generator 33 is configured to generate a signal having a waveform indicated by an equation (1) below, for example, as a first drive control signal for swinging the emission end portion of the fiber 12 for illumination in the X axis direction and output the signal to the D/A converter 34a, based on the control of the controller 25. Note that, in the equation (1) below, X(t) denotes a signal level at time t, Ax denotes an amplitude value independent of the time t, and G(t) denotes a predetermined function used in modulation of a sine wave sin(2πft).

X(t)=Ax×G(t)×sin(2πft) (1)

In addition, the signal generator 33 is configured to generate a signal having a waveform indicated by an equation (2) below, for example, as a second drive control signal for swinging the emission end portion of the fiber 12 for illumination in the Y axis direction and output the signal to the D/A converter 34b, based on the control of the controller 25. Note that, in the equation (2) below, Y(t) denotes the signal level at the time t, Ay denotes the amplitude value independent of the time t, G(t) denotes a predetermined function used in modulation of a sine wave sin(2πft+φ), and φ denotes a phase.

Y(t)=Ay×G(t)×sin(2πft+φ) (2)

The D/A converter 34a is configured to convert the digital first drive control signal outputted from the signal generator 33 to an analog drive signal DA and output the drive signal DA to the amplifier 35a.

The D/A converter 34b is configured to convert the digital second drive control signal outputted from the signal generator 33 to an analog drive signal DB and output the drive signal DB to the amplifier 35b.

The amplifier 35a is configured to amplify the drive signal DA outputted from the D/A converter 34a and output the amplified drive signal DA to the piezoelectric elements 15a and 15c of the actuator portion 15.

The amplifier 35b is configured to amplify the drive signal DB outputted from the D/A converter 34b and output the amplified drive signal DB to the piezoelectric elements 15b and 15d of the actuator portion 15.

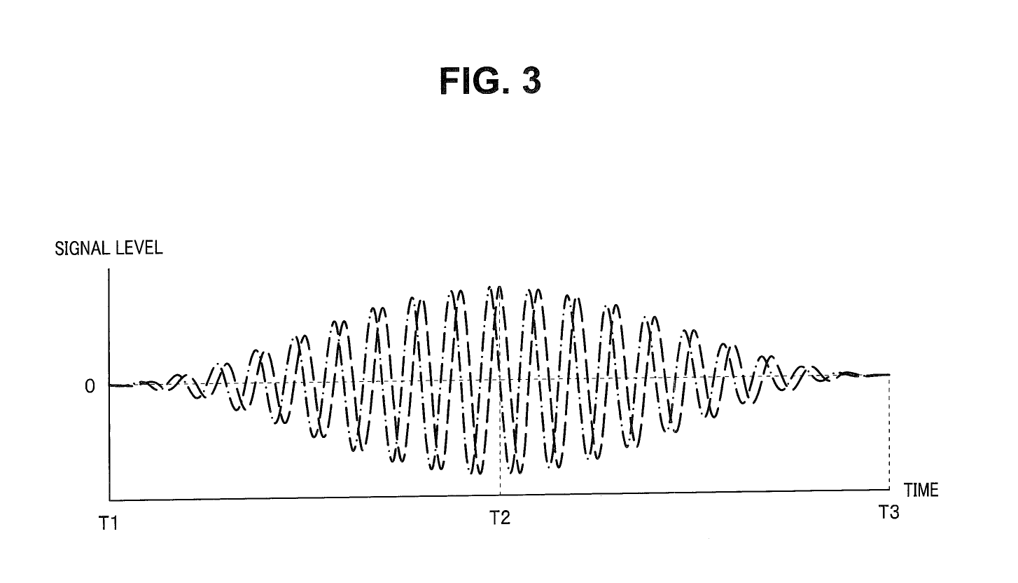

Here, for example, in the above-described equations (1) and (2), in a case that Ax=Ay and φ=π/2 are set, the drive voltage according to the drive signal DA having the signal waveform as illustrated by a broken line in FIG. 3 is applied to the piezoelectric elements 15a and 15c of the actuator portion 15, and the drive voltage according to the drive signal DB having the signal waveform as illustrated by a dashed line in FIG. 3 is applied to the piezoelectric elements 15b and 15d of the actuator portion 15. FIG. 3 is a diagram illustrating one example of the signal waveform of the drive signal supplied to the actuator portion.

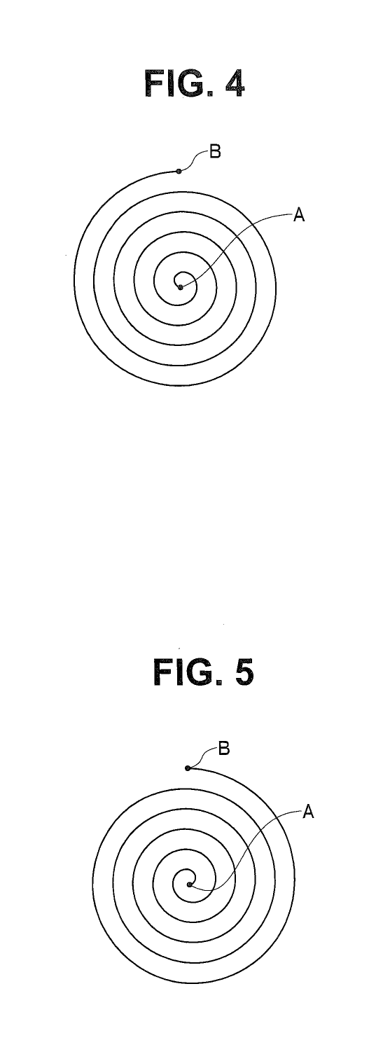

In addition, for example, in the case that the drive voltage according to the drive signal DA having the signal waveform as illustrated by the broken line in FIG. 3 is applied to the piezoelectric elements 15a and 15c of the actuator portion 15 and the drive voltage according to the drive signal DB having the signal waveform as illustrated by the dashed line in FIG. 3 is applied to the piezoelectric elements 15b and 15d of the actuator portion 15, the emission end portion of the fiber 12 for illumination is spirally swung, and a surface of the object is scanned along a spiral scanning route as illustrated in FIG. 4 and FIG. 5 according to such swinging. FIG. 4 is a diagram illustrating one example of the spiral scanning route from a center point A to an outermost point B. FIG. 5 is a diagram illustrating one example of the spiral scanning route from the outermost point B to the center point A.

More specifically, first, at time T1, the illumination light is radiated to a position corresponding to the center point A of the irradiation position of the illumination light on the surface of the object. Thereafter, as the signal level of the drive signals DA and DB increases from the time T1 to time T2, the irradiation position of the illumination light on the surface of the object is displaced to draw a first spiral scanning route to an outer side with the center point A as an origin, and further, when the time T2 comes, the illumination light is radiated to the outermost point B of the irradiation position of the illumination light on the surface of the object. Then, as the signal level of the drive signals DA and DB decreases from the time T2 to time T3, the irradiation position of the illumination light on the surface of the object is displaced to draw a second spiral scanning route to an inner side with the outermost point B as the origin, and further, when the time T3 comes, the illumination light is radiated to the center point A on the surface of the object.

That is, the actuator portion 15 includes the configuration capable of displacing the irradiation position of the illumination light emitted through the emission end portion to the object along the spiral scanning route illustrated in FIG. 4 and FIG. 5 by swinging the emission end portion of the fiber 12 for illumination based on the drive signals DA and DB supplied from the driver unit 22. In addition, the endoscope 2 includes the configuration capable of scanning the object by displacing the irradiation position of the illumination light to be radiated to the object.

The detection unit 23 has a function as a photodetection portion, and is configured to detect the return light received by the fiber 13 for light reception of the endoscope 2, and generate and successively output a photodetection signal according to intensity of the detected return light. More specifically, the detection unit 23 is configured including the photodetector 37, and an A/D converter 38.

The photodetector 37 includes an avalanche photodiode for example, and is configured to detect the light (return light) emitted from the light emission surface of the fiber 13 for light reception, generate an analog photodetection signal according to the intensity of the detected light, and successively output the signal to the A/D converter 38.

The A/D converter 38 is configured to convert the analog photodetection signal outputted from the photodetector 37 to a digital photodetection signal and successively output the signal to the controller 25.

In the memory 24, as control information used when controlling the main body device 3, for example, information of a parameter for specifying the signal waveform in FIG. 3, and a mapping table which is a table indicating a correspondence relation between output timing of the photodetection signal successively outputted from the detection unit 23 and a pixel position to be an application destination of pixel information obtained by converting the photodetection signal is stored.

In the memory 24, table data TD including a correction parameter for correcting the scanning width Wa of the endoscope 2 included in the endoscope information stored in the memory 16 is stored.

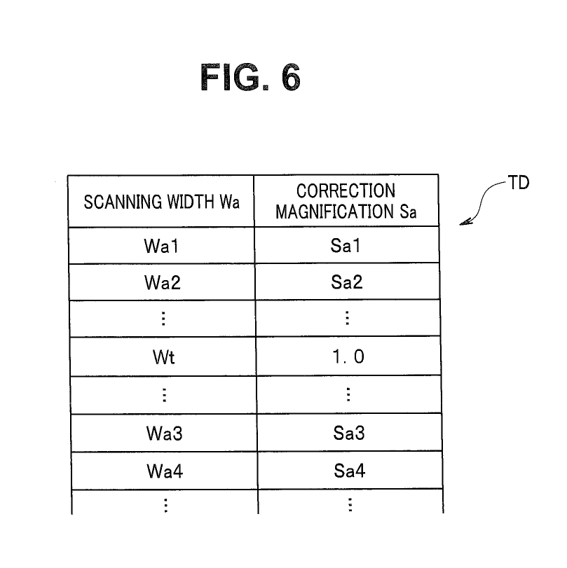

More specifically, the table data TD is, for example, as illustrated in FIG. 6, configured as data indicating the correspondence relation between the scanning width Wa of the endoscope 2 and a correction magnification Sa which is the correction parameter for correcting the scanning width Wa in accordance with a reference scanning width Wt. FIG. 6 is a diagram for describing an outline of the table data TD used in the processing of the endoscope processor relating to the embodiment.

Correction magnifications Sa1, Sa2, . . . included in the table data TD in FIG. 6 are calculated beforehand as values to be 1 in the case that the scanning width Wa is equal to the reference scanning width Wt, to be smaller than 1 in the case that the scanning width Wa is larger than the reference scanning width Wt, and to be larger than 1 in the case that the scanning width Wa is smaller than the reference scanning width Wt. In addition, the correction magnifications Sa1, Sa2, . . . included in the table data TD in FIG. 6 are calculated as values capable of making the scanning width Wa coincide or roughly coincide with the reference scanning width Wt by being multiplied with the known amplitude values Ax and Ay. Furthermore, the reference scanning width Wt is preset as a reference value of the scanning width of the endoscope 2 in a state that production tolerance and/or time degradation of the actuator portion 15 has not occurred.

In the memory 24, correction information to be used in magnification chromatic aberration correction processing for correcting a magnification chromatic aberration generated due to an optical characteristic of the illumination optical system 14 is stored.

More specifically, the above-described correction information includes a scaling rate SR for scaling an R image (to be described later) with a size of a G image (to be described later) as a reference and a scaling rate SB for scaling a B image (to be described later) with the size of the G image as the reference, for example.

The controller 25 includes an integrated circuit such as an FPGA (field programmable gate array), and is configured to perform an operation according to an operation of the input device 5. In addition, the controller 25 is configured to detect whether or not the insertion portion 11 is electrically connected to the main body device 3 by detecting a connection state of the connector portion 61 in the connector receiving portion 62 through a signal line or the like not shown in the figure. Furthermore, the controller 25 is configured to read the control information stored in the memory 24 when the power source of the main body device 3 is turned on and perform the operation according to the read control information. In addition, the controller 25 is configured including a light source control portion 25a, a scanning control portion 25b, a correction processing portion 25c, and an image processing portion 25d.

The light source control portion 25a is configured to perform the control for causing the R light, the G light and the B light to be repeatedly emitted in the order to the light source unit 21, for example, based on the control information read from the memory 24.

The scanning control portion 25b is configured to perform the control for causing the drive signals DA and DB having the signal waveform as illustrated in FIG. 3 to be generated to the driver unit 22, for example, based on the control information read from the memory 24. In addition, the scanning control portion 25b is configured to perform the control for causing the amplitude value Ax of the drive signal DA and the amplitude value Ay of the drive signal DB to be changed to the driver unit 22, based on the correction parameter obtained by the processing of the correction processing portion 25c.

The correction processing portion 25c is configured to perform the processing of acquiring the correction parameter for correcting the scanning width Wa of the endoscope 2 included in the endoscope information, based on the endoscope information read from the memory 16 of the endoscope 2 connected to the main body device 3 and the table data TD read from the memory 24. In addition, the correction processing portion 25c is configured to output the correction parameter obtained through the above-described processing to the scanning control portion 25b.

The image processing portion 25d is configured including an image generation portion 25m and an image correction portion 25n.

The image generation portion 25m is configured to generate the R image which is the image according to the return light of the R light radiated along the first spiral scanning route (the scanning route illustrated in FIG. 4), the G image which is the image according to the return light of the G light radiated along the first spiral scanning route, and the B image which is the image according to the return light of the B light radiated along the first spiral scanning route respectively by converting the photodetection signal successively outputted from the detection unit 23 within a period from the time T1 to T2 to the pixel information and mapping the pixel information, for example, based on the mapping table included in the control information read from the memory 24.

The image correction portion 25n is configured to perform the magnification chromatic aberration correction processing for correcting the magnification chromatic aberration among the R image, the G image and the B image generated by the image generation portion 25m, based on the correction information read from the memory 24. In addition, the image correction portion 25n is configured to generate an observation image by combining the R image, the G image and the B image to which the above-described magnification chromatic aberration correction processing is executed, and successively output the generated observation image to the display device 4.

More specifically, the image correction portion 25n is configured to perform the magnification chromatic aberration correction processing of scaling the R image generated by the image generation portion 25m at a magnification SR and scaling the B image generated by the image generation portion 25m at a magnification SB the scaling rates SR and SB included in the correction information read from the memory 24, for example.

The display device 4 includes an LCD (liquid crystal display) for example, and is configured to display the observation image outputted from the main body device 3.

The input device 5 is configured including one or more switches and/or buttons capable of instructing the controller 25 according to the operation by a user. Note that the input device 5 may be configured as a device separate from the main body device 3, or may be configured as an interface integrated with the main body device 3.

Next, the operation or the like of the endoscope system 1 including the configuration as described above will be described.

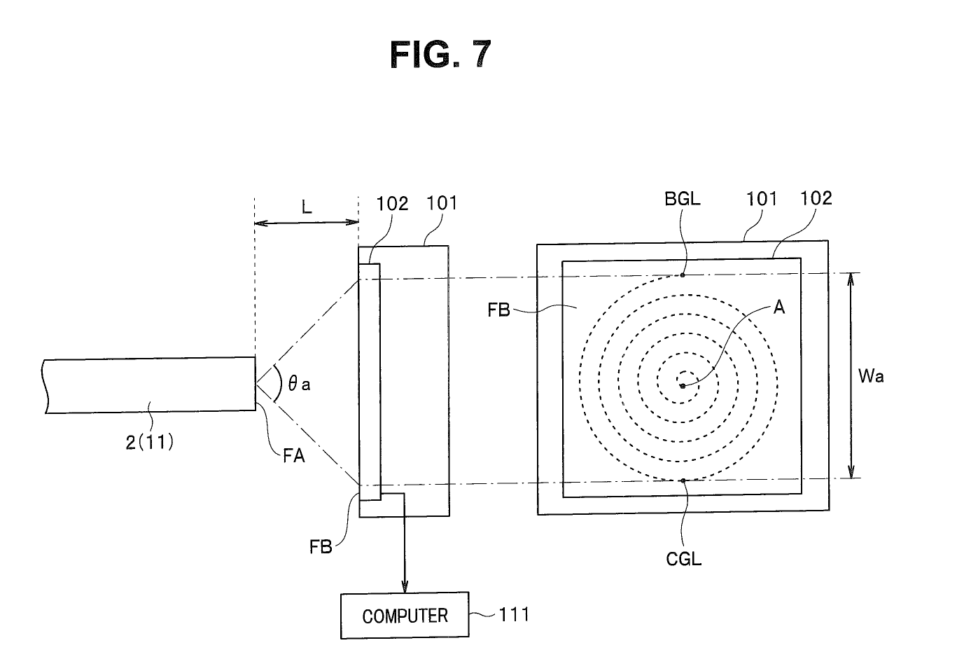

First, a specific example of a calculation method of the scanning width Wa stored in the memory 16 will be described while appropriately referring to FIG. 7. FIG. 7 is a diagram for describing the specific example of the calculation method of the scanning width of the endoscope.

For example, upon manufacturing or a delivery inspection of the endoscope 2, after connecting the respective portions of the endoscope system 1 and turning on the power source, as illustrated in FIG. 7, a factory operator arranges a distal end face FA of the endoscope 2 (insertion portion 11) and a light receiving surface FB of a position detection element (abbreviated as a PSD, hereinafter) 102 provided in an inspection jig 101 opposite to each other at a predetermined distance L, and wires a cable such that an output signal from the PSD 102 is inputted to a computer 111. Note that, in FIG. 7, for convenience of illustration and description, the distal end face FA of the endoscope 2 (insertion portion 11) and the light emission surface of the lens 14b are flush.

Thereafter, the factory operator gives the instruction for starting scanning for an inspection to the controller 25 by operating an inspection switch (not shown in the figure) of the input device 5, for example.

The light source control portion 25a performs the control for causing the G light to be intermittently generated to the light source unit 21, when detecting that the inspection switch of the input device 5 is operated. In addition, the scanning control portion 25b performs the control for causing the drive signal DA having the amplitude value Ax and the drive signal DB having the amplitude value Ay to be generated to the driver unit 22, when detecting that the inspection switch of the input device 5 is operated.

Then, according to the control of the light source control portion 25a and the scanning control portion 25b as described above, the light receiving surface FB of the PSD 102 is scanned by the pulsed G light emitted through the distal end face FA of the endoscope 2 (insertion portion 11), and position information indicating the irradiation position of the G light radiated to the light receiving surface FB along the spiral scanning route is outputted from the PSD 102 to the computer 111. Note that it is assumed that the position information outputted from the PSD 102 to the computer 111 includes information capable of specifying the irradiation position of the G light radiated to the light receiving surface FB of the PSD 102 as a coordinate value of a rectangular coordinate system, for example.

The computer 111 acquires an irradiation position BGL (see FIG. 7) of the G light corresponding to an outermost point of the spiral scanning route and an irradiation position CGL (see FIG. 7) of the G light opposing the outermost point on an outermost periphery of the spiral scanning route respectively, based on the position information outputted from the PSD 102. Then, the computer 111 calculates a distance between the irradiation position BGL and the irradiation position CGL as the scanning width Wa of the endoscope 2.

That is, in the present embodiment, the scanning width Wa of the endoscope 2 calculated by the method illustrated above is stored in the memory 16. In addition, according to the method illustrated above, the scanning width Wa of the endoscope 2 is stored in the memory 16 as a parameter indicating a scanning range of the spiral scanning route. Note that the computer 111 may be an arithmetic unit provided outside the main body device 3, or may be an arithmetic unit built in the main body device 3.

Next, a specific example of the operation performed in the controller 25 will be described.

After connecting the respective portions of the endoscope system 1 and turning on the power source, a user such as an operator gives the instruction for starting scanning for observation to the controller 25 by operating an observation start switch (not shown in the figure) of the input device 5.

The correction processing portion 25c reads the endoscope information from the memory 16 of the endoscope 2 connected to the main body device 3 when the power source of the main body device 3 is turned on. Then, the correction processing portion 25c performs the processing of acquiring the correction parameter for correcting the scanning width Wa of the endoscope 2 included in the endoscope information based on the endoscope information read from the memory 16 of the endoscope 2 and the table data TD read from the memory 24, and outputs the acquired correction parameter to the scanning control portion 25b.

More specifically, the correction processing portion 25c performs the processing of acquiring the correction magnification Sa corresponding to the scanning width Wa of the endoscope 2 included in the endoscope information read from the memory 16 by referring to the table data TD as illustrated in FIG. 6, for example, and outputs the acquired correction magnification Sa to the scanning control portion 25b. That is, according to such processing of the correction processing portion 25c, for example, in the case that the scanning width included in the endoscope information read from the memory 16 is Wa2, the correction magnification Sa2 is acquired as the correction parameter for correcting the scanning width Wa2, and the correction magnification Sa2 is outputted to the scanning control portion 25b.

The light source control portion 25a performs the control for causing the R light, the G light and the B light to be repeatedly emitted in the order to the light source unit 21, when detecting that the observation start switch of the input device 5 is operated.

The scanning control portion 25b calculates a new amplitude value CAx by multiplying the amplitude value Ax by the correction magnification Sa obtained by the processing of the correction processing portion 25c, and calculates a new amplitude value CAy by multiplying the amplitude value Ay by the correction magnification Sa. Then, the scanning control portion 25b performs the control for causing the drive signal DA having the amplitude value CAx and the drive signal DB having the amplitude value CAy to be generated to the driver unit 22, when detecting that the observation start switch of the input device 5 is operated. Note that the amplitude values CAx and CAy described above are held until the power source of the main body device 3 is turned off, for example.

That is, the scanning control portion 25b causes the R image, the G image and the B image generated by the image generation portion 25m to be uniformly scaled according to the correction magnification Sa, by increasing or decreasing the amplitude values Ax and Ay of the drive signal supplied to the endoscope 2 for displacing the irradiation position of the illumination light guided by the fiber 12 for illumination along the spiral scanning route according to the correction magnification Sa.

The image generation portion 25m generates the R image, the G image and the B image according to the photodetection signal successively outputted from the detection unit 23 respectively, based on the mapping table included in the control information read from the memory 24.

The image correction portion 25n performs the magnification chromatic aberration correction processing of scaling the R image generated by the image generation portion 25m at the magnification SR and scaling the B image generated by the image generation portion 25m at the magnification SB using the scaling rates SR and SB included in the correction information read from the memory 24. In addition, the image correction portion 25n generates the observation image by combining the R image, the G image and the B image to which the above-described magnification chromatic aberration correction processing is executed, and successively outputs the generated observation image to the display device 4.

That is, the image correction portion 25n performs the magnification chromatic aberration correction processing for correcting the magnification chromatic aberration among the R image, the G image and the B image uniformly scaled according to the correction magnification Sa. In addition, the image correction portion 25n performs the processing of scaling the R image and the B image scaled according to the correction magnification Sa, with the size of the G image scaled according to the correction magnification Sa as the reference, as the magnification chromatic aberration correction processing.

According to the operation of the controller 25 as described above, the scanning width when scanning the object by radiating the light of three colors that are the R light, the G light and the B light along the spiral scanning route can be made to coincide or roughly coincide with the reference scanning width Wt. Therefore, according to the operation of the controller 25 as described above, the magnification chromatic aberration among the images of the three colors can be corrected in the state that dispersion of the size among the images of the three colors that are the R image, the G image and the B image generated due to the production tolerance and/or the time degradation or the like of the actuator portion 15, that is, a generation factor of excessive correction and correction insufficiency of the magnification chromatic aberration, is eliminated. As a result, according to the present embodiment, the magnification chromatic aberration in the images acquired using the scanning type endoscope can be corrected appropriately (in proper quantities).

In addition, according to the present embodiment, the magnification chromatic aberration among the images of the three colors can be corrected appropriately (in proper quantities) without storing the plurality of scaling rates SR and the plurality of scaling rates SB for responding to the dispersion of the size among the images of the three colors that are the R image, the G image and the B image generated due to the production tolerance and/or the time degradation or the like of the actuator portion 15 in the memory 24, for example. As a result, according to the present embodiment, a capacity of a storage medium such as the memory can be prevented from being oppressed due to many parameters to be used in the magnification chromatic aberration correction processing being stored in the storage medium.

Note that, according to the present embodiment, for example, in the case that the predetermined distance L is known, a view angle θa (see FIG. 7) of the endoscope 2 calculated based on the predetermined distance L and the scanning width Wa of the endoscope 2 may be stored in the memory 16, and the table data TE indicating the correspondence relation between the view angle θa and a correction magnification Sc which is a correction parameter for correcting the view angle θa in accordance with a reference view angle θt may be stored in the memory 24. That is, in such a case, the view angle θa of the endoscope 2 is stored in the memory 16 as the parameter indicating the scanning range of the spiral scanning route. Note that the reference view angle θt is assumed to be preset as a reference value of the view angle of the endoscope 2 in the state that the production tolerance and/or the time degradation of the actuator portion 15 has not occurred.

In addition, according to the present embodiment, for example, in the case that the table data TD is stored in the computer 111 used upon the manufacturing or the delivery inspection of the endoscope 2, the correction magnification Sa corresponding to the scanning width Wa of the endoscope 2 may be stored in the memory 16. Furthermore, in such a case, for example, the scanning control portion 25b may read the endoscope information from the memory 16 and calculate the amplitude values CAx and CAy using the correction magnification Sa included in the read endoscope information. That is, in the case that the correction magnification Sa corresponding to the scanning width Wa of the endoscope 2 is stored in the memory 16, at least some functions of the correction processing portion 25c may be incorporated in the scanning control portion 25b.

In addition, according to the present embodiment, the correction magnification Sa acquired by the processing of the correction processing portion 25c may be outputted to the image correction portion 25n, for example, instead of being outputted to the scanning control portion 25b. Furthermore, in such a case, for example, the image correction portion 25n may perform the magnification chromatic aberration correction processing of uniformly scaling the R image, the G image and the B image at a magnification Sa and then further scaling the R image, which is scaled at the magnification Sa, at the magnification SR and scaling the B image, which is scaled at the magnification Sa, at the magnification SB.

Furthermore, according to the present embodiment, for example, in the case that a shift direction of a disposing position of the fiber 12 for illumination to the center of the actuator portion 15 is known, the image correction portion 25n may further perform pixel position correction processing of moving the pixel position of the R image scaled at the magnification SR in accordance with the pixel position of the G image and moving the pixel position of the B image scaled at the magnification SB in accordance with the pixel position of the G image. Note that, in the case of performing such pixel position correction processing, preferably, table data capable of respectively specifying a relative moving amount of the pixel position of the R image to the pixel position of the G image and a relative moving amount of the pixel position of the B image to the pixel position of the G image according to four to eight shift directions may be stored in the memory 24.

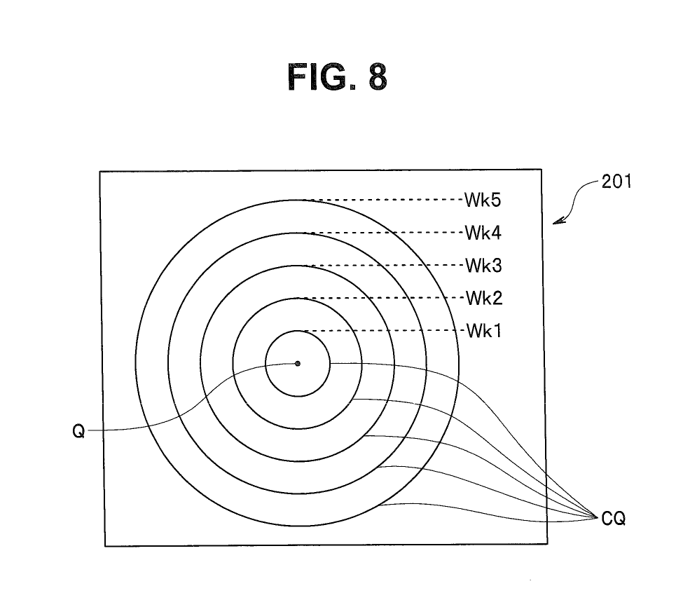

On the other hand, according to the present embodiment, regardless of calculation of the scanning width Wa based on the position information obtained when the light receiving surface FB of the PSD 102 is scanned, for example, the scanning width Wa may be acquired based on the R image, the G image and the B image generated by the image generation portion 25m when a test chart 201 as illustrated in FIG. 8 is scanned. FIG. 8 is a diagram illustrating one example of a test chart available when acquiring the scanning width of the endoscope.

On a surface of the test chart 201, as illustrated in FIG. 8, a plurality of circles CQ centering on a center point Q are concentrically drawn. In addition, on the surface of the test chart 201, scanning width Wk1, Wk2, . . . corresponding to respective diameters of the plurality of circles CQ are written.

Here, a specific example of work using the test chart 201 will be described below. Note that, below, specific description relating to a part to which the already-described operation or the like is applicable is appropriately omitted. In addition, below, the case that five circles CQ centering on the center point Q are concentrically drawn on the surface of the test chart 201, five scanning widths Wk1 to Wk5 corresponding to the respective diameters of the five circles CQ are written on the surface of the test chart 201, and the five scanning widths Wk1 to Wk5 satisfy the relation of Wk1<Wk2<Wk3=Wt<Wk4<Wk5 will be described as an example.

For example, in a facility of a destination of the endoscope 2, a maintenance operator confirms that the respective portions of the endoscope system 1 are connected and the power source of the respective portions of the endoscope system 1 is supplied, and then arranges the distal end face FA of the endoscope 2 (insertion portion 11) and the surface of the test chart 201 opposite to each other at the predetermined distance L. In addition, when arranging the distal end face FA of the endoscope 2 (insertion portion 11) and the surface of the test chart 201 opposite to each other, the maintenance operator performs positioning for matching the center point A of the spiral scanning route with the center point Q of the plurality of circles CQ.

Thereafter, the maintenance operator gives the instruction for starting scanning for a simple inspection to the controller 25 by operating a simple inspection switch (not shown in the figure) of the input device 5, for example.

When detecting that the simple inspection switch of the input device 5 is operated, the light source control portion 25a performs the control for causing the R light, the G light and the B light to be repeatedly emitted in the order to the light source unit 21. In addition, when detecting that the simple inspection switch of the input device 5 is operated, the scanning control portion 25b performs the control for discarding the amplitude values CAx and CAy that are already held and causing the drive signal DA having the amplitude value Ax and the drive signal DB having the amplitude value Ay to be generated to the driver unit 22.

Then, according to the control of the light source control portion 25a and the scanning control portion 25b as described above, the plurality of circles CQ drawn on the surface of the test chart 201 are scanned by the light of the three colors that are the R light, the G light and the B light emitted through the distal end face FA of the endoscope 2 (insertion portion 11), and the photodetection signal according to the return light of the light of the three colors is successively outputted from the detection unit 23.

On the other hand, when detecting that the simple inspection switch of the input device 5 is operated, the image processing portion 25d performs the operation for generating an inspection image for the simple inspection by combining the R image, the G image and the B image generated in the image generation portion 25m and successively outputting the generated inspection image to the display device 4.

Then, according to the operation of the image processing portion 25d as described above, the image before being scaled according to the correction magnification Sa, the scaling rate SR and the scaling rate SB is displayed at the display device 4 as the inspection image. In addition, according to the operation of the image processing portion 25d as described above, for example, in the case that the scanning width of the endoscope 2 connected to the main body device 3 is smaller than the reference scanning width Wt, the inspection image not including the three circles CQ corresponding to the scanning widths Wk3 to Wk5 is displayed at the display device 4. Furthermore, according to the operation of the image processing portion 25d as described above, for example, in the case that the scanning width of the endoscope 2 connected to the main body device 3 is equal to or larger than the reference scanning width Wt, the inspection image including at least the three circles CQ corresponding to the scanning widths Wk1 to Wk3 is displayed at the display device 4.

The maintenance operator specifies a scanning width Wkd (d=1, 2, 3, 4 or 5) corresponding to a circle CQD on the outermost side included in the inspection image by visually confirming the inspection image displayed at the display device 4. In addition, the maintenance operator roughly calculates how far an outermost portion of the inspection image is separated from the circle CQD and acquires a rough estimate correction value Cd by visually confirming the inspection image displayed at the display device 4. Thereafter, the maintenance operator calculates the (rough) scanning width Wa of the endoscope 2 by adding the rough estimate correction value Cd to the scanning width Wkd, and performs the operation for inputting the calculated scanning width Wa to the controller 25 in the input device 5.

Then, according to work using the test chart 201 as described above, the processing for acquiring the correction magnification Sa corresponding to the scanning width Wa inputted according to the operation of the input device 5 is performed in the correction processing portion 25c, the processing for calculating the new amplitude values CAx and CAy according to the correction magnification Sa is performed in the scanning control portion 25b, and also the drive signal DA having the amplitude value CAx and the drive signal DB having the amplitude value CAy are outputted from the driver unit 22. Thus, also in the case of acquiring the scanning width Wa through the work using the test chart 201, effects almost similar to the effects in the case of acquiring the scanning width Wa through the work using the inspection jig 101 can be demonstrated.

On the other hand, by appropriately modifying the configuration of the main body device 3 of the present embodiment, the configuration may be adapted to the endoscope including the object optical system that obtains an optical image of the object and an image pickup device such as a CCD or a CMOS that picks up the optical image of the object.

More specifically, for example, the three images that are the R image, the G image and the B image according to the optical image of the object picked up by the image pickup device of the endoscope may be generated in the image generation portion 25m, the processing for scaling the three images and making the three images coincide with a predetermined reference size may be performed in the correction processing portion 25c, and the magnification chromatic aberration correction processing for correcting the magnification chromatic aberration among the three images scaled to the predetermined reference size may be performed in the image correction portion 25n.

Note that it is needless to say that the present invention is not limited to each embodiment described above and various changes and applications are possible without deviating from the gist of the invention.

What is claimed is:

1. An endoscope processor used in combination with a scanning type endoscope capable of scanning an object by displacing an irradiation position of illumination light to be radiated to the object, the endoscope processor comprising:
an image generation portion configured to respectively generate a plurality of color images according to return light of the illumination light radiated to the object;
a correction processing portion configured to perform processing of acquiring a correction magnification for correcting a scanning width or a view angle of the scanning type endoscope in accordance with a reference value; and
an image correction portion configured to perform magnification chromatic aberration correction processing for correcting a magnification chromatic aberration among the plurality of color images uniformly scaled according to the correction magnification.

2. The endoscope processor according to claim 1, further comprising
a scanning control portion configured to uniformly scale the plurality of color images generated by the image generation portion according to the correction magnification by increasing or decreasing an amplitude value of a drive signal supplied to the scanning type endoscope in order to displace the irradiation position of the illumination light along a predetermined scanning route according to the correction magnification.

3. The endoscope processor according to claim 1,
wherein the image correction portion uniformly scales the plurality of color images generated by the image generation portion according to the correction magnification and then performs the magnification chromatic aberration correction processing.

4. The endoscope processor according to claim 1,
wherein the scanning width of the scanning type endoscope is calculated based on position information indicating the irradiation position of the illumination light obtained when a position detection element is scanned as the object.

5. The endoscope processor according to claim 1,
wherein the scanning width of the scanning type endoscope is acquired based on the plurality of color images generated by the image generation portion when a predetermined test chart is scanned as the object.

6. The endoscope processor according to claim 1,
wherein the image correction portion performs processing of scaling images other than a predetermined color image scaled according to the correction magnification with a size of the predetermined color image scaled according to the correction magnification as a reference, as the magnification chromatic aberration correction processing.
